(19) United States
(12) Patent Application Publication
(10) Pub. No.: US 2008/0175975 A1
(43) Pub. Date: Jul. 24, 2008

(54) Method For Producing a Dha-Containing Fatty Acid Composition

(75) Inventor: Dirk Fabritius, Ansbach (DE)

Correspondence Address: HAMMER & HANF, PC, 3125 SPRINGBANK LANE, SUITE G, CHARLOTTE, NC, 28226, US

(73) Assignee: NUTRINOVA NUTRITION SPECIALTIES & FOOD INGREDIENTS GMBH, Frankfurt am Main (DE)

(21) Appl. No.: 11/814,291

(22) PCT Filed: Jan. 26, 2006
(86) PCT No.: PCT/EP2006/000677

§ 371 Date: Aug. 7, 2007

(30) Foreign Application Priority Data
Jan. 26, 2005 (DE) 10 2005 003 625.2

(51) Int. Cl. A23D 7/00; C11B 1/04

(52) U.S. Cl. 426/601; 554/8

(57) ABSTRACT

The invention relates to a method for producing a fatty acid composition which, based on the entire weight of the fatty acids and/or fatty acid derivatives contained in the fatty acid composition, contains at least 70.0% by weight of docosahexaenoic acid and/or docosahexaenoic alkyl ester. The inventive method comprises the following steps: a) transesterifying a biomass obtained from Ulkenia sp. with an alcohol, thereby producing at least one docosahexaenoic alkyl ester and at least one saturated fatty acid ester, b) producing a solution which contains urea, at least a part of the biomass from step a) and at least one organic solvent, c) cooling or concentrating the solution from step b), thereby producing i) a precipitate which contains urea and at least a part of the saturated fatty acid esters, and ii) a liquid fraction, and d) separating the precipitate i) from the liquid fraction ii). The invention also relates to the fatty acid composition obtainable according to the inventive method and to the use thereof.

The present invention relates to a method for producing a fatty acid composition which, based on the total weight of the fatty acids and/or fatty acid derivatives contained in the fatty acid composition, contains at least 70.0% by weight of docosahexaenoic acid and/or docosahexaenoic acid alkyl ester.

Long-chain polyunsaturated fatty acids (PUFAs) are essential fatty acids in human metabolism. PUFAs can be subdivided into two large groups. In addition to the group of ω-6 PUFAs which are formulated proceeding from linoleic acid, there is the group of ω-3 PUFAs which are made up starting from α-linolenic acid.

PUFAs are important building blocks of cell membranes, the retina and the meninges and precursors of important hormones, for example prostaglandins, thromboxanes and leukotrienes.

In addition to the function as building blocks, in the course of recent years it has increasingly been found that PUFAs directly have multiple beneficial effects on the human organism or diseases.

A multiplicity of clinical studies have found that PUFAs can make an important contribution to healing or alleviation, for example in the case of cancer, rheumatic arthritis, high blood pressure and neurodermatitis and many other diseases. In these cases the use of docosahexaenoic acid (DHA; all-cis-4,7,10,13,16,19-docosahexaenoic acid) esters (in contrast to free DHA) is frequently particularly advantageous, because such esters (in particular the ethyl esters and triglycerides) have a tendency to have a pleasant taste and to be readily absorbed by the digestive system. These findings were originally responsible for the fact that international institutions and authorities have delivered recommendations which control the daily intake of PUFAs.

PUFAs cannot be synthesized de-novo by humans, since they lack the enzyme systems which can introduce a double bond into the carbon chain at positions >C9 (lack of Δ12-desaturase). Humans are only able to synthesize polyunsaturated fatty acids via the supply of precursor fatty acids (for example α-linolenic acid) from the diet. However, whether this amount is sufficient to cover the requirement of polyunsaturated fatty acids is contested.

The great majority of essential fatty acids are taken in via the diet. In particular vegetable oils are enriched with ω-6 fatty acids (for example evening primrose oil contains γ-linolenic acid (GLA)) but only up to a chain length of C18, and fish oils and oils from microorganisms, with ω-3 fatty acids (for example salmon oil contains eicosapentaenoic acid (EPA) and docosahexaenoic acid (DHA; all-cis-4,7,10,13,16,19-docosahexaenoic acid)). In principle, fish oils and oils from microorganisms are the only commercial sources of polyunsaturated fatty acids. Generally, however, the content of the desired PUFAs is low and they are present in a mixture, in which case PUFAs acting antagonistically can also be present. In order to consume the recommended daily dose of PUFAs, therefore a high quantity of oil must be consumed. In particular, this applies to those patients who must consume high doses of PUFAs (for example in the case of cystic fibrosis). To achieve an effect of the individual PUFAs in as targeted a manner as possible, enriched or high-purity PUPAs must be used. Therefore, in the prior art, there is a great requirement for high-purity PUFAs.

Numerous methods have been used individually or in combination to isolate (or at least concentrate) and recover certain fatty acids and their derivatives from a multiplicity of naturally occurring sources. These methods include fractional crystallization at low temperatures, molecular distillation, urea adduct crystallization, extraction with metal salt solutions, supercritical fluid fractionation on countercurrent columns and HPLC methods.

In W. W. Christie, Lipid Analysis, pp. 147-149 (Pergamon Press, 1976), a general method is disclosed in which use is made of urea in order to separate off methyl esters of saturated fatty acids from a mixture which also contains methyl esters of polyunsaturated fatty acids. According to Christid, when urea is crystallized in the presence of a plurality of different long-chain aliphatic compounds, hexagonal crystals are formed which include the aliphatic compounds (what are termed urea complexes). The aliphatic compounds can then be readily separated off from the solution by filtration.

Christie states in general that the methyl esters of saturated fatty acids more readily form urea complexes than the methyl esters of unsaturated fatty acids of the same chain length and that the methyl esters of unsaturated fatty acids having trans double bonds more readily form urea complexes than the methyl esters of the corresponding unsaturated fatty acids having cis double bonds. Christie also describes the use of urea crystallization for concentrating methyl esters of polyunsaturated fatty acids from a mixture which contains methyl esters of polyunsaturated fatty acids and methyl esters of saturated fatty acids. In this manner, apparently fatty acid compositions having a mass yield of 20% can be obtained, but, in the publication, however, specific data on experimental procedure and PUFA yield are lacking. In addition, no data on the quality of the products, for example the peroxide content, can be inferred from it.

A further publication which describes separating off fatty acid methyl esters with the use of urea crystallization is T. Nakahara, T. Yokochi, T. Higashihara, S. Tanaka, T. Yaguchi, D. Honda “Production of Docosahexaenoic and Docosapentaenoic Acids by Schizochytrium sp. isolated from Yap Islands”, JAOCS, volume 73, No. 11, pp. 1421-26 (1996). Nakahara et al. describe the production of a mixture of fatty acid methyl esters by washing and drying Schizochytrium sp. cells and then directly performing methyl esterification with methanol in the presence of 10% strength HCl. Nakahara et al. report that 34.9% of the resultant methyl esters contained DHA residues and 8.7% of the resultant methyl esters contained DPA residues. To concentrate these polyunsaturated fatty acid esters, methanol and urea are added to the mixture. The mixture is then heated to 60° C. in order to dissolve the urea and subsequently cooled to 10° C. to crystallize out the urea. According to Nakahara et al., in this manner a mixture is obtained which contains 73.3% DHA methyl esters and 17.7% DPA methyl esters. Further details on experimental procedure, yield and quality of the resultant products cannot be taken from the publication.

WO 01/51598 A1 discloses a method for producing an enriched mixture of polyunsaturated fatty acid esters in which an oil of Schizochytrium sp. is transesterified with an alcohol (methanol). The fatty acid esters are then dissolved in a medium together with urea and cooled or concentrated in order to separate off at least in part the saturated fatty acid esters which precipitate out together with the urea. In this manner an oil can be obtained which, according to gas chromatography, contains 23.4% by weight of ω-6 DPA methyl ester, 65.2% by weight of ω-3 DHA methyl ester, 2.9% by weight of myristic acid methyl ester and 1.5% by weight of palmitic acid methyl ester.

A disadvantage of the above-described procedures is, in particular, the use of the toxic methanol for esterifying the fatty acids. Therefore, the fatty acid compositions described in these publications are not suitable for uses in the food sector. In addition, the disclosure of the methods, in particular of the method of Christie and of the method of Nakahara et al., is full of gaps and incomplete such that they cannot be reproduced. Furthermore, in particular for the method described in Nakahara et al., it must be assumed that owing to the relatively high elevation of the DHA content, a comparatively low overall yield is obtained.

The growing use of polyunsaturated fatty acids, particularly DHA, and of esters thereof in medicine and nutrition gives rise to the desire for a method which is as cost-efficient and reliable as possible for producing a fatty acid composition having a fraction which is as high as possible of polyunsaturated fatty acids, in particular docosahexaenoic acid and/or docosahexaenoic acid alkyl ester, and simultaneously a fraction which is as low as possible of saturated fatty acids.

In the light of this prior art, it was therefore an object of the present invention to provide such a method. In this case, the method according to the invention should permit the production of the fatty acid composition in the simplest possible manner, on a large scale and cost-effectively.

At the same time, the method should deliver a fatty acid composition having the highest possible purity and quality, in particular having an acid number as low as possible and/or having a heavy metal content as low as possible.

Furthermore, the method should be as gentle as possible, and in particular lead to fatty acid compositions having a peroxide content as low as possible.

In addition, the method according to the invention should be able to be carried out as far as possible using solvents which are as food-safe as possible. In particular, the use of substances which are hazardous to health should be avoided as far as possible.

The fatty acid compositions obtainable by the method should have an ethyl carbamate content as low as possible, in particular in order to enable the use of the fatty acid compositions in the food sector without concern.

These and other objects which, although they are not mentioned explicitly, may be derived as obvious from the context discussed herein or inevitably result therefrom, are achieved by a method having all the features of the present claim 1. Expedient modifications of the method according to the invention are described in the subclaims referred back to claim 1. The claims of the product category protect the fatty acid composition obtainable by the method according to the invention and the use claims indicate particularly advantageous fields of use of the fatty acid composition according to the invention.

By providing a method for producing a fatty acid composition which, based on the total weight of the fatty acids and/or fatty acid derivatives contained in the fatty acid composition, contains at least 70.0% by weight of all-cis-4,7,10,13,16,1,9-docosahexaenoic acid and/or all-cis-4,7,10,13,16,19-docosahexaenoic acid allyl ester, wherein:
a) a biomass obtainable from Ulkenia sp. is transesterified with at least one alcohol to form at least one docosahexaenoic acid alkyl ester and at least one saturated fatty acid ester,
b) a solution is produced which contains urea, at least a part of the transesterified biomass from step a) and at least one organic solvent,
c) the solution from step b) is cooled or concentrated to form
   i) a precipitate which contains urea and at least a part of the saturated fatty acid ester, and
   ii) a liquid fraction,
d) the precipitate i) is separated off from the liquid fraction ii),
   in a manner which is not readily predictable, a novel and useful process is successfully provided for producing a fatty acid composition which, based on the total weight of the fatty acids and/or fatty acid derivatives contained in the fatty acid composition contains at least 70.0% by weight of docosahexaenoic acid and/or docosahexaenoic acid alkyl ester. This was surprising, in particular, because the enrichment in particular of the DHA and/or the DHA-ester is achieved according to the invention, although the compounds to be separated according to the invention are very complex molecules and there are only minimal structural differences between them.

At the same time, the procedure according to the invention has a number of further advantages:
The process according to the invention can be carried out in a simple manner, on a large scale and cost effectively.
The process according to the invention is exceedingly efficient and makes the fatty acid compositions accessible with comparatively high yields.
The process according to the invention delivers a fatty acid composition of comparatively high purity and quality, in particular having a comparatively low acid number and a comparatively low heavy metal content. The acid number of the fatty acid composition, measured as specified in AOCS Official Method Ja 8-87, is preferably less than or equal to 1.5 mg of KOH per g of fatty acid composition, more expediently less than or equal to 0.8 mg of KOH per g of fatty acid composition, more preferably less than or equal to 0.2 mg of KOH per g of fatty acid composition, in particular less than or equal to 0.06 mg of KOH per g of fatty acid composition.
The method according to the invention is relatively mild and leads to fatty acid compositions having a relatively low peroxide content. The peroxide value of the fatty acid composition, measured as specified in AOCS Official Method Cd-3d 63, is preferably less than or equal to 0.5 meq per kg of fatty acid composition, expediently less than or equal to 0.1 meq per kg of fatty acid composition, more preferably less than or equal to 0.05 meq per kg of fatty acid composition, in particular less than or equal to 0.01 meq per kg of fatty acid composition. The heavy metal content of the fatty acid composition, measured as specified in LMGB [German Food and Food-Contact Commodities Act] paragraph 35 L06.00-7, is preferably less than or equal to 0.7 mg per kg of fatty acid composition, expediently less than or equal to 0.4 mg per kg of fatty acid composition, more preferably less than or equal to 0.3 mg per kg of fatty acid composition, in particular less than or equal to 0.2 mg per kg of fatty acid composition.
The fatty acid compositions obtainable by the method are distinguished by a relatively low cadmium content. The cadmium content of the fatty acid compositions obtainable according to the invention, measured as specified in LMBG paragraph 35 L06.00-7, is preferably less than or equal to 0.20 mg per kg of fatty acid composition, expediently less than or equal to 0.10 mg per kg of fatty acid composition, more preferably less than or equal to 0.05 mg per kg of fatty acid composition, in particular less than or equal to 0.03 mg per kg of fatty acid composition.
The fatty acid compositions obtainable by the method are distinguished by a relatively low lead content. The lead content of the fatty acid compositions obtainable according to the invention measured as specified in LMBG paragraph 35 L06.00-7, is preferably less than or equal to 0.20 mg per kg of fatty acid composition, expediently less than or equal to 0.10 mg per kg of fatty acid composition, more preferably less than or equal to 0.05 mg per kg of fatty acid composition, in particular less than or equal to 0.03 mg per kg of fatty acid composition.
The fatty acid compositions obtainable by the method are distinguished by a relatively low mercury content. The mercury content of the fatty acid compositions obtainable according to the invention, measured as specified in LMBG paragraph 35 L06.00-7, is preferably less than or equal to 0.10 mg per kg of fatty acid composition, expediently less than or equal to 0.05 mg per kg of fatty acid composition, more preferably less than or equal to 0.01 mg per kg of fatty acid composition, in particular less than or equal to 0.005 mg per kg of fatty acid composition.
The fatty acid compositions obtainable by the method are distinguished by a relatively low arsenic content. The arsenic content of the fatty acid compositions obtainable according to the invention, measured as specified in LMBG paragraph 35 L06.00-7, is preferably less than or equal to 0.20 mg per kg of fatty acid composition, expediently less than or equal to 0.10 mg per kg of fatty acid composition, more preferably less than or equal to 0.05 mg per kg of fatty acid composition, in particular less than or equal to 0.03 mg per kg of fatty acid composition.
The fatty acid compositions obtainable by the method are distinguished by a relatively low copper content. The copper content of the fatty acid compositions obtainable according to the invention, measured as specified in LMBG paragraph 35 L06.00-7, is preferably less than or equal to 0.25 mg per kg of fatty acid composition, expediently less than or equal to 0.20 mg per kg of fatty acid composition, more preferably less than or equal to 0.10 mg per kg of fatty acid composition, in particular less than or equal to 0.06 mg per kg of fatty acid composition.
The fatty acid compositions obtainable by the method are distinguished by a relatively low iron content. The iron content of the fatty acid compositions obtainable according to the invention, measured as specified in LMBG paragraph 35 L06.00-7, is preferably less than or equal to 0.25 mg per kg of fatty acid composition, expediently less than or equal to 0.20 mg per kg of fatty acid composition, more preferably less than or equal to 0.10 mg per kg of fatty acid composition, in particular less than or equal to 0.06 mg per kg of fatty acid composition.
The fatty acid compositions obtainable by the method are distinguished by a relatively low nickel content. The nickel content of the fatty acid compositions obtainable according to the invention, measured as specified in LMBG paragraph 35 L06.00-7, is preferably less than or equal to 0.25 mg per kg of fatty acid composition, expediently less than or equal to 0.20 mg per kg of fatty acid composition, more preferably less than or equal to 0.10 mg per kg of fatty acid composition, in particular less than or equal to 0.06 mg per kg of fatty acid composition.
In the method according to the invention, use is made of comparatively food-safe solvents.
The fatty acid compositions obtainable by the method according to the invention in addition have a comparatively low ethyl carbamate content and are therefore suitable, in particular, for applications in the food sector.

The present invention relates to a method for producing a fatty acid composition which, based on the total weight of the fatty acids and/or fatty acid derivatives contained in the fatty acid composition, preferably based on the total weight of the fatty acids and/or fatty acid esters contained in the fatty acid composition, in particular based on the total weight of the fatty acids and/or fatty acid triglycerides contained in the fatty acid composition, contains at least 70.0% by weight of all-cis-4,7,10,13,16,19-docosahexaenoic acid and/or all-cis-4,7,10,13,16,19-docosahexaenoic acid alkyl ester.

The expression “fatty acid composition” comprises in this context not only compositions which contain free fatty acids, but also compositions which contain fatty acid derivatives, preferably fatty acid esters, in particular fatty acid triglycerides, the fatty acid radicals being able in principle to be identical or different.

Fatty acids designate according to the invention aliphatic carboxylic acids which can be saturated or monounsaturated or polyunsaturated and preferably have 6 to 30 carbon atoms.

In the method according to the invention, as starting material, use is made of a biomass obtainable from Ulkenia sp. Biomasses obtainable from Ulkenia sp. are known per se. According to the invention, use can be made not only of biomasses from Ulkenia sp. wild type strains but also biomasses of mutant or recombinant Ulkenia sp. strains which produce DHA (all-cis-4,7,10,13,16,19-docosahexaenoic acid) and/or DPA (all-cis-4,7,10,13,16,19-docosapentaenoic acid) efficiently. Such mutant or recombinant strains include microorganisms which, compared with the percentage of the original Ulkenia sp. wild type strain, using the same substrate, contain a higher percentage of DHA and/or DPA in fats and/or, compared with the amount produced by the original Ulkenia sp. wild type strain, contain a higher overall amount of the lipids, using the same substrate.

According to a particularly preferred embodiment of the present invention, as starting material, use is made of an Ulkenia sp. dry matter. According to a further preferred embodiment of the present invention, an oil from Ulkenia sp. is used as starting material.

Oils from Ulkenia sp. are expediently obtained by culturing the microorganism, which is rich in DHA, harvesting the biomass from the culture, disintegrating it and isolating the oil. A very particularly expedient method in this context is described in WO 03/033631 A1, the contents of the disclosure of which are hereby explicitly incorporated herein by reference.

For isolation of the oil, preferably use is made of extraction methods with organic solvents, in particular hexane, or with supercritical liquids.

Expediently, the oil is extracted from the biomass by percolation of the dried biomass with hexane. Such extractions with organic solvents are described, inter alia, in WO 9737032, WO 9743362 and EP 515460. A particularly extensive account may also be found in the Journal of Dispersion Science and Technology 10, 561-579, 1989 “Biotechnological Processes for the Production of PUFAs”.

Alternatively, the extraction can also proceed without solvent. A method which is particularly expedient in this context is described in EP-A-1178118. In this method, a solvent is avoided by producing an aqueous suspension of the biomass and separating off the oil phase from the aqueous phase by centrifugation.

The composition of the biomass obtainable from Ulkenia sp. can vary within a broad range. Preferably it contains at least one glyceride, in particular a triglyceride, which comprises at least one polyunsaturated fatty acid radical. According to a particularly preferred embodiment, at least 10%, particularly preferably at least 25%, and in particular at least 30%, of the fatty acid radicals in the biomass are DHA radicals.

A “glyceride” is, as far as the expression is used herein, an ester of glycerol and at least one fatty acid, in which case one to three hydroxyl groups of the glycerol have been esterified with one or more fatty acid radicals. If a plurality of fatty acid radicals are present, the fatty acid radicals can be identical or different.

In many suitable starting materials, the majority of the glycerides are triglycerides, that is to say esters of three fatty acid radicals and glycerol. In this case each fatty acid radical can either be saturated (that is to say all bonds between the carbon atoms are single bonds) or unsaturated (that is to say there is at least one carbon-carbon double or triple bond). The type of the unsaturated fatty acid radicals is occasionally characterized herein by a c. This number indicates the position of the first double bond, counting, starting from the terminal methyl group of the fatty acid or the fatty acid radical.

According to the invention the biomass obtainable from Ulkenia sp. is first transesterified with at least one alcohol. The purpose of the transesterification step is elimination of the fatty acid radicals from the glycerol backbone of the glycerides in the starting material and formation of separate esters of each of the radicals (at least one docosaliexaenoic acid alkyl ester and at least one saturated fatty acid ester), so that the esters can be separated from one another.

According to the invention the transesterification preferably proceeds with use of at least one alcohol of the formula R¹—OH, wherein R¹ is a linear or branched alkyl radical having 1 to 20, preferably 1 to 6, in particular 1 to 4, carbon atoms. Particular preference is given to the methyl esters and ethyl esters, and in particular the ethyl esters.

According to a first preferred embodiment of the invention, the transesterification is catalyzed by at least one base. Preferred bases comprise sodium methoxide, potassium methoxide, elemental sodium, sodium hydroxide and potassium hydroxide. Preferably, the volumetric ratio of the biomass to the base/alcohol mixture is 1:1 to 1:5. The concentration of the base in the alcohol is preferably 0.1 to 2 M. According to a preferred variant, the transesterification reaction is carried out at room temperature (that is to say at a temperature in the range of approximately 20-25° C.) for 6-20 hours.

According to a further preferred variant, the transesterification reaction is carried out at a temperature above room temperature, preferably at a temperature of at least 40° C., particularly preferably at a temperature of 70 to 150° C., in particular at a temperature above the boiling point of one or more components in the mixture (under reflux).

According to a second preferred embodiment of the invention, the transesterification is catalyzed by at least one acid by preferably incubating the biomass at a temperature of approximately 0 to approximately 150° C. in a mixture which the at least one alcohol and at least one acid, preferably HCl, preferably under an inert gas atmosphere, and in the absence of water.

According to a preferred variant, the triglyceride/acid/alcohol mixture is refluxed for at least 2 hours. According to a further preferred variant, the triglyceride/acid/alcohol mixture is held at a temperature of 0 to 50° C. for at least 12 hours.

Since the acid-catalyzed transesterification is customarily reversible, the alcohol is preferably charged in a large excess, so that the reaction essentially proceeds up to complete conversion. Preferably, the triglyceride concentration in the alcohol/acid mixture is 0.1 to 15% by weight. The concentration of the acid, preferably HCl, in the alcohol/acid mixture is preferably 4 to 15% by weight. Such a mixture can be produced by many methods known in the prior art, such as, for example, by introducing gaseous hydrogen chloride into dry alcohol, or by addition of acetal chloride to alcohol. Although HCl is most preferred according to the invention, other acids can alternatively be used. Such an acid is H₂SO₄, which is preferably used at a concentration of 0.5 to 5% by weight in the alcohol. However, consideration should be given to the fact that H₂SO₄ is a strongly oxidizing agent and is therefore preferably only used in combination with short reflux times (that is to say less than 6 hours), at low concentrations (that is to say less than 5% by weight) and at low temperatures (that is to say below 150° C.).

A further example of a suitable acid is boron fluoride, which is preferably used at a concentration of 1-20% by weight. However, HCl is preferred to boron fluoride, because boron fluoride has a greater tendency to form unwanted by-products.

The transesterification reaction preferably proceeds under an inert gas atmosphere (for example noble gas and/or N₂). In addition, an antioxidant (for example ascorbyl palmitate or propyl galate) can also be added to the reaction mixture, in order to prevent autooxidation.

During the transesterification, preferably at least one organic solvent is added. Preferred solvents comprise, in particular, those compounds which are able to dissolve the fatty acid esters to be transesterified. When the starting material contains a plurality of fatty acid esters to be transesterified, the organic solvent is preferably able to dissolve all of the fatty acid esters to be transesterified. Solvents which are very particularly suitable according to the invention comprise dichloromethane, acetonitrile, ethyl acetate and diethyl ether, in particular dichloromethane.

After the transesterification, the esters are preferably separated off from the reaction mixture by addition of water. Frequently the esters (which are organic) float at the top on the reaction mixture and can be separated off simply from the remaining reaction mixture. This applies in particular to large scale industrial applications.

Alternatively, a liquid-liquid solvent extraction can be used in order to separate off the esters from the remaining reaction mixture. This extraction can vary in a broad range. According to a preferred variant, water is added to the mixture and the esters are extracted with a nonpolar solvent. If the transesterification was catalyzed by at least one base, the water preferably comprises a sufficient amount of acid, preferably HCl, citric acid or acetic acid, in particular HCl, in order to neutralize the mixture, or particularly preferably to give the mixture a weakly acid pH. The ratio of the total volume of the nonpolar solvent to the volume of the reaction mass (including the added water) can also be varied within a broad range and is particularly from 1:3 to 4:3. According to a particularly preferred embodiment, the mixture is extracted with a plurality of fractions of the nonpolar organic solvent which are combined at the end. Nonpolar solvents which are particularly suitable according to the invention include petroleum ether, pentane, hexane, cyclohexane and heptane, with hexane and petroleum ether being most preferred.

The nonpolar solvent can also contain a small amount of a weakly polar organic solvent such as, for example, diethyl ether. The use of such a polar component has a tendency to lead to an improvement of the extraction of the fatty acid esters from the aqueous layer, because such esters are likewise weakly polar. If a weakly polar, organic component is used, the volumetric concentration of the weakly polar component to the nonpolar component is preferably not greater than approximately 20%, particularly preferably not greater than 10%, and in particular 5% to 10%.

The resultant organic extraction solvent layer can be washed in order, for example, to remove any acid residues and/or remaining water. Acid residues are preferably removed by washing the layer with an aqueous solution which contains a weak base, for example potassium carbonate. The remaining water can be removed, for example, by washing the layer with a brine (that is to say a saturated salt solution) and/or by drying with an anhydrous salt (for example sodium sulfate or magnesium sulfate).

After the extraction, the fatty acid esters can be concentrated in the nonpolar solvent layer. According to a preferred embodiment of this invention, the esters are concentrated by evaporating a part of the nonpolar solvent.

Transesterification of a biomass obtainable from Ulkenia sp., in addition to the DHA alkyl ester, customarily delivers other fatty acid esters. Many of these fatty acid esters, in particular the saturated fatty acid esters, have unknown and/or disadvantageous medical properties and nutritional properties. It is therefore necessary to remove in particular the saturated fatty acid esters as completely as possible from the transesterification reaction mixture. The method according to the invention therefore comprises a urea crystallization in which
b) a solution is first produced which contains urea, at least a part of the transesterified biomass from step a) and at least one organic solvent,
c) the solution from step b) is cooled or concentrated to form
   i) a precipitate which contains urea and at least a part of the saturated fatty acid ester, and
   ii) a liquid fraction,
d) the precipitate i) is separated off from the liquid fraction ii).

When urea is crystallized in a solution which contains polyunsaturated fatty acid esters (for example esters of DHA) and saturated fatty acid esters, which were obtained by transesterification using the above-described method, a precipitate forms which contains the urea and at least a part of the saturated fatty acid esters. This precipitate, however, comprises a substantially lower fraction of the polyunsaturated fatty acid esters than the solution. The majority of the polyunsaturated fatty acid esters therefore remains in solution and can readily be separated off from the precipitated saturated fatty acid esters.

The urea crystallization separation method of the invention comprises first forming a solution which contains the fatty acid esters and urea. The amount of urea is preferably proportional to the total amount of the saturated fatty acids which are to be separated off from the solution. The mass ratio of the mixture of the fatty acid esters to the urea is preferably 1:1 to 1:4.

The solution preferably also comprises at least one organic solvent which dissolves urea and the desired DHA ester, particularly preferably urea and all fatty acid esters in the mixture. Solvents which are particularly suitable in this context include alcohols having 1 to 4 carbon atoms, with methanol and ethanol, in particular ethanol, being particularly preferred. The volumetric ratio of the mixture of the fatty acid esters to the solvent is preferably 1:5 to 1:20.

Preferably essentially all of the urea is dissolved in the solution. This can generally be achieved by heating the solution, preferably to a temperature above 50° C. According to a very particularly preferred embodiment of the invention, the solution is prepared by dissolving the urea and the fatty acid ester mixture in the solution separately from one another, preferably with heating, in particular to temperatures above 50° C., and then mixing the resultant solutions with one another.

To separate off the saturated fatty acid esters, the solution containing the fatty acid esters and the urea is preferably cooled to form a urea-comprising precipitate. Preferably, the solution is cooled to a temperature below 40° C., preferably below or equal to 30° C., in particular below or equal to 25° C., with the temperature advantageously being above 110° C., preferably above or equal to 15° C., expediently above or equal to 20° C. The cooled solution is preferably allowed to stand with occasional stirring at the cooled temperature for a certain period of time, typically no longer than approximately 20 hours, preferably for 5 to 20 hours.

According to a further preferred embodiment of this invention, a urea-containing precipitate is formed by concentrating the solution containing the fatty acid esters and the urea. The solution can be concentrated, for example, by evaporating a part of the solvent in the solution. The amount of solvent removed is preferably sufficient to effect a urea concentration in the solution which exceeds the saturation concentration.

The urea crystallizatin is expediently carried out under an inert gas atmosphere (for example noble gases and/or N₂).

After the urea-containing precipitate has formed, the precipitate is preferably separated off from the liquid fraction which is enriched with polyunsaturated esters. This is preferably achieved by filtration or centrifugation. According to a particularly preferred embodiment, the precipitate is thereafter washed with a small amount of the organic solvent (preferably saturated with urea), in order to recover polyunsaturated fatty acid esters adhering to the precipitate. This wash solution is in turn preferably combined with the liquid fraction.

The liquid fraction is preferably concentrated, combined with water, and the esters contained in the liquid fraction are preferably extracted with a nonpolar solvent. The liquid fraction can be concentrated, for example by evaporating a part of the solvent from the liquid fraction, the amount of the solvent evaporated preferably being not so great that further urea precipitates. The amount of water which is added to the concentrated liquid fraction can vary within a wide range. Preferably the volumetric ratio of water to the concentrated liquid fraction is 4:1 to 1:1.

In the context of a very particularly preferred embodiment, a sufficient amount of acid, preferably HCl, to neutralize the urea is also added. For the purposes of the present invention, particularly suitable nonpolar solvents comprise petroleum ether, pentane, hexane, cyclohexane, ethyl acetate and heptane, with hexane being most preferred. The volumetric ratio of the nonpolar solvent to the concentrated liquid fraction/water mixture is preferably 1:5 to 5:1.

According to a particularly preferred embodiment of the present invention, the liquid fraction is also extracted with a weakly polar organic solvent in order to maximize the recovery of the fatty acid esters (which, as noted above, are weakly polar). Weakly polar solvents which are particularly suitable according to the invention include diethyl ether and ethyl acetate, with diethyl ether being most preferred. Preferably, the volumetric ratio of the weakly polar solvent to the concentrated liquid fraction/water mixture is 1:5 to 5:1. After the extraction with the weakly polar solvent, the extracts are preferably combined.

After the extraction, the extracts can be dried, for example by washing with a brine and/or using an anhydrous salt (for example sodium sulfate). The solution is then preferably concentrated, for example by partial or complete evaporation of the solvent.

The method according to the invention is distinguished, in particular, by an exceedingly efficient removal of the saturated fatty acid esters. Therefore, in the context of the present invention, the transesterified biomass is preferably subjected to the urea crystallization directly, that is to say without further intermediate steps.

Although it is not generally necessary, it is also possible, before the urea crystallization, to increase the fraction of polyunsaturated fatty acids in the transesterified biomass by partial removal of the other components, in order in this manner to increase still further the efficiency of the method according to the invention. This can proceed in a manner known per se, in which case the use of extraction methods, in particular extraction with nonpolar solvents (as described above), and also winterization methods, being particularly well proven.

Winterization comprises cooling a solution which contains the transesterified biomass to a temperature which causes at least a part of the saturated fatty acid esters to precipitate, while a substantially smaller fraction of the polyunsaturated fatty acid esters precipitates. Preferably, the solution is cooled to a temperature below 0° C., particularly preferably to a temperature in the range from −30 to −10° C., in particular to a temperature in the range from −25 to −15° C. The solution is preferably held at these temperatures for up to 20 hours and under an inert gas atmosphere.

The winterization is preferably carried out in an organic solvent which dissolves the DHA ester and at least one saturated fatty acid ester in the fatty acid ester mixture. Particularly suitable solvents include methanol and ethanol, with ethanol being most preferred. Preferably, the volumetric ratio of the fatty acid ester mixture to the organic solvent is 1:5 to 1:20.

After formation of the precipitate, the solution is preferably separated off from the precipitate to form a liquid fraction which is enriched in the desired polyunsaturated fatty acid esters. This is preferably achieved by filtration or centrifugation. After the liquid fraction is separated off, it is expediently concentrated by evaporating the solvent in a rotary evaporator.

Possible fields of application of the fatty acid compositions obtainable according to the invention are immediately obvious to those skilled in the art. They are suitable, in particular, for all applications which are indicated for PUFAs and PUFA esters. In this case the fatty acid compositions according to the invention can mostly be used directly. However, for some applications it is necessary to saponify in advance the fatty acid ester or fatty acid esters in the liquid phase. This can be achieved, for example, by reaction with KOH in ethanol.

The fatty acid compositions obtainable according to the invention are used, in particular, as active ingredient or component in pharmaceutical fatty acid compositions, as component in cosmetics preparations, as food additive or food ingredient, as a component of functional foods and for producing highly concentrated PUFA secondary products, such as esters and acids.

The invention will be described in more detail hereinafter by examples, without restricting the inventive concept hereby.

### EXAMPLE 1

### 1. Transesterification

A solution of 13.12 g of Na ethylate in 228 g of absolute ethanol is added dropwise with stirring to a mixture of 560.5 g of Ulkenia sp. crude oil and 292 g of absolute ethanol. The resultant mixture is stirred for 2.5 hours. Thereafter, 4466 g of water are added and the batch is allowed to stand for one hour. After 1 hour, a further 171 g of water are added. The batch is allowed to stand for a further 12 hours, whereupon two phases form. The oil phase is separated off and ethanol and water residues are removed on a rotary evaporator. 405.7 g of transesterified ethyl ester oil are obtained.

### 2. Urea Precipitation

680 g of urea are dissolved in 4430 ml of ethanol at 77° C. (6 liter four-neck round-bottom flask equipped with stirrer, thermometer and cooler). In parallel, 404 g of transesterified ethyl ester oil in 443 ml of ethanol are preincubated at 70° C. and added to the urea solution. The batch is allowed to stand for 12 hours. The precipitate formed is separated off and the remaining liquid phase is concentrated to 1.5 liters on a rotary evaporator. Thereafter, 1.5 liters of 2 molar hydrochloric acid and 2.5 liters of water are added to the liquid phase. The organic phase is separated off and dried at 45° C. on the vacuum pump.

230 g of purified oil are obtained. The fatty acid profile of the oil is given in Table 1 and the characteristic data on oil quality (acid number, peroxide value, heavy metal content) are given in Table 2.

### EXAMPLE 2

### 1. Transesterification

1 kg of Ulkenia sp. dry biomass are stirred with 2.5 liters of 10% strength ethanolic sulfuric acid at 75° C. under nitrogen for 48 hours. The batch is cooled to 50° C. and extracted with 3.5 liters of hexane. The hexane phase is separated off and the solvent (hexane) is removed on a rotary evaporator. 390.1 g of transesterified ethyl ester oil are obtained.

### 2. Urea Precipitation

599 g of urea are dissolved in 3.9 liters of ethanol at 77° C. (6 liter four-neck round-bottom flask equipped with stirrer, thermometer and cooler). In parallel, 390 g of transesterified ethyl ester oil in 390 ml of ethanol are preincubated at 70° C. and added to the urea solution. The batch is allowed to stand for 12 hours. The precipitate formed is separated off and the remaining liquid phase is concentrated to 1.5 liters on a rotary evaporator. Thereafter, 1.5 liters of 2 molar hydrochloric acid and 2.5 liters of water are added to the liquid phase. The organic phase is separated off and dried at 45° C. on a vacuum pump. 216.2 g of purified oil are obtained. The fatty acid profile of the oil is given in Table 1 and the characteristic data on oil quality (acid number, peroxide value, heavy metal content) are given in Table 2.

TABLE 1
Fatty acid profile of the purified oil (data in % by weight)

| | Example 1 | Example 2 |
|---|---|---|
| Myristic acid | 0.0 | 1.0 |
| Pentadecanoic acid | 0.0 | 0.2 |
| Palmitic acid | 0.3 | 1.6 |
| Heptadecanoic acid | 0.0 | 0.7 |
| Stearic acid | 0.5 | 0.4 |
| Eicosatetraenoic acid (ω-7) | 1.3 | 1.4 |
| Eicosatetraenoic acid (ω-3) | 1.2 | 1.1 |
| Docosapentaenoic acid (ω-6) | 17.2 | 15.9 |
| Docosapentaenoic acid (ω-3) | 0.3 | 1.0 |
| Docosahexaenoic acid | 75.4 | 71.4 |
| Other fatty acids | 3.9 | 5.4 |

TABLE 2
Quality of the purified oil

| | Example 1 | Example 2 |
|---|---|---|
| Acid number¹ [mg of KOH/g] | 0.06 | 0.80 |
| Peroxide value² [meq/kg] | 0.0 | 0.0 |
| Cadmium³ [mg/kg] | <0.03 | <0.03 |
| Lead³ [mg/kg] | <0.03 | <0.03 |
| Mercury³ [mg/kg] | <0.002 | <0.002 |
| Arsenic³ [mg/kg] | <0.03 | <0.03 |
| Copper³ [mg/kg] | <0.06 | <0.06 |
| Iron³ [mg/kg] | <0.06 | <0.18 |
| Nickel³ [mg/kg] | <0.06 | <0.06 |

¹measured as specified in AOCS Official Method Ja 8-87
²measured as specified in AOCS Official Method Cd-3d 63 (American Oil Chemists Society)
³measured as specified in LMBG paragraph 35 L06.00-7 (German Food and Food Contact Commodities Act)

1. A method for producing a fatty acid composition which, based on the total weight of the fatty acids and/or fatty acid derivatives contained in the fatty acid composition, contains at least 70.0% by weight of docosahexaenoic acid and/or docosahexaenoic acid alkyl ester, wherein:
a) a biomass obtainable from Ulkenia sp. is transesterified with at least one alcohol to form at least one docosahexaenoic acid alkyl ester and at least one saturated fatty acid ester,
b) a solution is produced which contains urea, at least a part of the transesterified biomass from step a) and at least one organic solvent,
c) the solution from step b) is cooled or concentrated to form
i) a precipitate which contains urea and at least a part of the saturated fatty acid ester, and
ii) a liquid fraction,
d) the precipitate i) is separated off from the liquid fraction ii).

2. The method as claimed in claim 1, characterized in that, as biomass, use is made of an oil from Ulkenia sp.

3. The method as claimed in claim 1, characterized in that, as biomass, use is made of Ulkenia sp. dry biomass.

4. The method as claimed in claim 1, characterized in that, for the transesterification, use is made of an alcohol of the formula R¹—OH, R¹ being a linear or branched alkyl radical having 1 to 20 carbon atoms.

5. The method as claimed in claim 1, characterized in that the transesterification is carried out in the presence of at least one base.

6. The method as claimed in claim 1, characterized in that the trans-esterification is carried out in the presence of at least one acid.

7. The method as claimed in claim 1, characterized in that the organic solvent from step b) comprises at least one alkyl alcohol having 1 to 4 carbon atoms.

8. The method as claimed in claim 1, characterized in that, in step c), the solution is cooled to a temperature not above or equal to 15° C.

9. The method as claimed in claim 8, characterized in that the solution is cooled to a temperature in the range from 15° C. to 25° C.

10. The method as claimed in claim 1, characterized in that, in step b), the transesterified biomass from step a) is used directly.

11. The method as claimed in claim 1, characterized in that, in step b), use is made of an oil which is obtained from the transesterified biomass from step a) by elevating the fraction of polyunsaturated fatty acids in the transesterified biomass by partially separating off the other components.

12. The method as claimed in claim 11, characterized in that the fraction of polyunsaturated fatty acids in the transesterified biomass is elevated by extraction methods.

13. The method as claimed in claim 11, characterized in that the fraction of polyunsaturated fatty acids in the transesterified biomass is elevated by winterizing methods.

14. The method as claimed in claim 1, characterized in that the fatty acid ester or fatty acid esters is or are saponified in the liquid phase.

15. A fatty acid composition obtainable by claim 1.

16. The fatty acid composition as claimed in claim 15, characterized in that it has an acid number, measured as specified in AOCS Official Method Ja 8-87, of less than or equal to 1.5 mg of KOH per g of fatty acid composition.

17. The fatty acid composition as claimed in claim 15, characterized in that it has a peroxide value, measured as specified in AOCS Official Method Cd-3d 63, of less than or equal to 0.5 meq per kg of fatty acid composition.

18. The fatty acid composition as claimed in claim 15, characterized in that it has a heavy metal content, measured as specified in LMBG paragraph 35 L06.00-7, of less than or equal to 0.7 mg per kg of fatty acid composition.

19. The use of a fatty acid composition as claimed in claim 15 as active ingredient or component in pharmaceutical compositions.

20. The use of a fatty acid composition as claimed in claim 15 as component in cosmetics preparations.

21. The use of a fatty acid composition as claimed in claim 15 as food additive and/or as food ingredient.

22. The use of a fatty acid composition as claimed in claim 15 as component of animal feed.
